(19) United States
(12) Patent Application Publication
(10) Pub. No.: US 2023/0357756 A1
(43) Pub. Date: Nov. 9, 2023

(54) COMPOSITIONS, METHODS, AND SYSTEMS FOR CELL LABELING

(71) Applicant: Washington University, St. Louis, MO (US)

(72) Inventors: Samantha Morris, St. Louis, MO (US); Kunal Jindal, St. Louis, MO (US)

(73) Assignee: Washington University, St. Louis, MO (US)

(21) Appl. No.: 18/312,940

(22) Filed: May 5, 2023

Related U.S. Application Data
(60) Provisional application No. 63/338,748, filed on May 5, 2022.

(51) Int. Cl. C12N 15/10

(52) U.S. Cl. CPC C12N 15/1065; C12N 15/1096

(57) ABSTRACT

Compositions, methods, and systems for labeling cells to track cell lineage within single-cell transcriptomic, genomic, epigenomic, and/or multi-omics assays are disclosed.

### CROSS-REFERENCE TO RELATED APPLICATIONS

This application claims the benefit of priority to U.S. Provisional Application Ser. No. 63/338,748 filed on May 5, 2022, the content of which is incorporated by reference herein in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to compositions, methods, and systems for labeling cells to capture cell lineage within single-cell transcriptomic, genomic, epigenomic, and/or multi-omics assays.

### BACKGROUND OF THE INVENTION

Single-cell RNA sequencing has revolutionized the study of biology. More recently, single-cell measurements have been extended to multiple cellular modalities like chromatin state, DNA conformation, and spatial arrangement of cells. Single-cell measurement of cell lineage along with transcriptomic state has been previously developed. But measurement of lineage along with other single-cell phenotypes has lagged behind, often forcing the users to choose between multi-omics and lineage analysis in their system of interest.

### SUMMARY OF THE INVENTION

Among the various aspects of the present disclosure is the provision of compositions and methods for labeling cells to track cell lineage within single-cell transcriptomic, genomic, epigenomic, and/or multi-omics assays.

Briefly, therefore, the present disclosure is directed to compositions of genetic constructs and methods of use thereof.

In one aspect. a genetic construct configured to label cells to capture cell lineage within at least one single-cell state assay is disclosed that includes a reporter gene with modifications in the 3′ UTR. The modifications include a lineage tracing barcode, first and second flanking sequences at the 3′ and 5′ ends of the lineage tracing barcode, respectively; and a reverse transcription priming site at the 5′ end of the second flanking sequence. The lineage tracing barcode includes a static random sequence configured to uniquely label single cells and associated clones. In some aspects, the first and second flanking sequences each comprises a transposase. In some aspects, the first and second flanking sequences each comprises a Nextera adapter. In some aspects, the at least one single-cell state assay is selected from a single-cell transcriptomic assay, a genomic assay, an epigenomic assay, a multi-omics assay, and any combination thereof. In some aspects, the genetic construct is packaged into a lentiviral particle. In some aspects, the genetic construct further includes a promoter sequence positioned at the 3′ end of the first flanking sequence. In some aspects, the reporter gene is a green fluorescent protein (GFP) gene.

In other aspects, a method of labeling cells to trace cell lineage within at least one single-cell state assay is disclosed that includes inserting a genetic construct into the genome of a cell. The genetic construct is configured to label cells to capture cell lineage within at least one single-cell state assay, The genetic construct includes a reporter gene with modifications in the 3′ UTR that include a lineage tracing barcode, first and second flanking sequences at the 3′ and 5′ ends of the lineage tracing barcode, respectively, and a reverse transcription priming site at the 5′ end of the second flanking sequence. The lineage tracing barcode includes a static random sequence configured to uniquely label single cells and associated clones. In some aspects, the genetic construct is inserted into the genome of the cells by viral transduction. In some aspects, the cell lineage is traced using scRNA-seq or scATAC-seq lineage tracing. In some aspects, the first and second flanking sequences each comprises a transposase. In some aspects, the first and second flanking sequences each comprises a Nextera adapter. In some aspects, the at least one single-cell state assay is selected from a single-cell transcriptomic assay, a genomic assay, an epigenomic assay, a multi-omics assay, and any combination thereof. In some aspects, the genetic construct is packaged into a lentiviral particle. In some aspects, the genetic construct further comprises a promoter sequence positioned at the 3′ end of the first flanking sequence. In some aspects, the reporter gene of the genetic construct is a green fluorescent protein (GFP) gene.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic of a genetic construct (CellTag-multi) used in lineage tracing assays in accordance with one aspect of the disclosure.

FIG. 1B is a schematic of a genetic construct (CellTag-multiB) used in lineage tracing assays in accordance with another aspect of the disclosure.

FIG. 2A is a workflow diagram of the lineage tracing analysis process using the genetic construct of FIG. 1A in accordance with an aspect of the disclosure.

FIG. 2B is a workflow diagram of the lineage tracing analysis process using the genetic construct of FIG. 1B in accordance with another aspect of the disclosure.

FIG. 3 is a schematic diagram illustrating various parameters used to establish cell identity.

FIG. 4 is a workflow diagram of a CellTag-ATAC-RNA lineage tracing assay.

FIG. 5 contains maps summarizing RNA cells and ATAC cells of two different clones identified using the CellTag-ATAC-RNA lineage tracing assay of FIG. 4.

FIG. 6 is a workflow diagram showing the identification of state-fate relationships in hematopoiesis using the CellTag-ATAC-RNA lineage tracing assay of FIG. 4.

FIG. 7 contains maps summarizing cell state-fate relationships in hematopoiesis obtained using the CellTag-ATAC-RNA lineage tracing assay of FIG. 4.

FIG. 8 contains a heat map summarizing the ATAC profiles of reprogrammed iEP cells obtained using the CellTag-ATAC-RNA lineage tracing assay of FIG. 4.

FIG. 9A is a graph illustrating the relatively high proportion of reprogrammed iEP cells within a first clone cluster identified using the CellTag-ATAC-RNA lineage tracing assay of FIG. 4.

FIG. 9B is a graph illustrating the relatively high proportion of dead-end iEP cells within a second clone cluster identified using the CellTag-ATAC-RNA lineage tracing assay of FIG. 4.

### DETAILED DESCRIPTION OF THE INVENTION

In various aspects, a DNA construct is disclosed that permanently labels cells with combinations of heritable nucleic acid barcodes (CellTags) and molecular biology workflows that allow parallel measurement of cell phenotype and lineage relationships. In some aspects, modifications of the DNA construct are disclosed that are compatible with a wide range of single-cell assays. The DNA construct design, along with the custom molecular biology workflows, ensures compatibility with single-cell assays based on the capture of poly-adenylated RNA, tagged fragments of cDNA, tagged fragments of DNA, or any combination thereof, providing for lineage capture in single-cell transcriptomic, genomic, epigenomic and multi-omics assays.

Single-cell RNA sequencing has revolutionized the study of biology. More recently, single-cell measurements have been extended to multiple cellular modalities like chromatin state, DNA conformation, and spatial arrangement of cells. Single-cell measurement of cell lineage along with transcriptomic state has been previously developed. But measurement of lineage along with other single-cell phenotypes has lagged behind, often forcing the users to choose between multi-omics and lineage analysis in their system of interest. With the technology of the present disclosure, a flexible lineage tracing solution that allows the adaptation of lineage tracing to a wide array of current and future single-cell assays is described.

CellTagging is a straightforward system for lineage tracing. As disclosed herein, the DNA construct extends the lineage tracing aspect of CellTagging to a wide range of single-cell assays. In some embodiments, the method of cell labeling makes use of CellTag-multi, a DNA construct suitable for scRNA-seq and scATAC-seq lineage tracing. In other aspects, the method of cell labeling makes use of CellTag-multiB, a DNA construct suitable for assays including, but not limited to, single-cell histone profiling (e.g. single-cell CUT&Tag) that typically require incubating intact nuclei with primary antibodies overnight, likely leading to a significant loss of nuclear RNA (and hence CellTag RNA) due to diffusion. In other additional aspects, this construct can be applied to other single-cell assays with some modification in the capture protocol. In general, the CellTag-multi lineage tracing system consists of 3 components: (1) the lineage tracing construct itself, (2) a modified library preparation protocol to allow CellTag capture in a wide variety of single-cell genomics assays, and (3) a computational pipeline that allows identification of clones across single-cell data from multiple modalities.

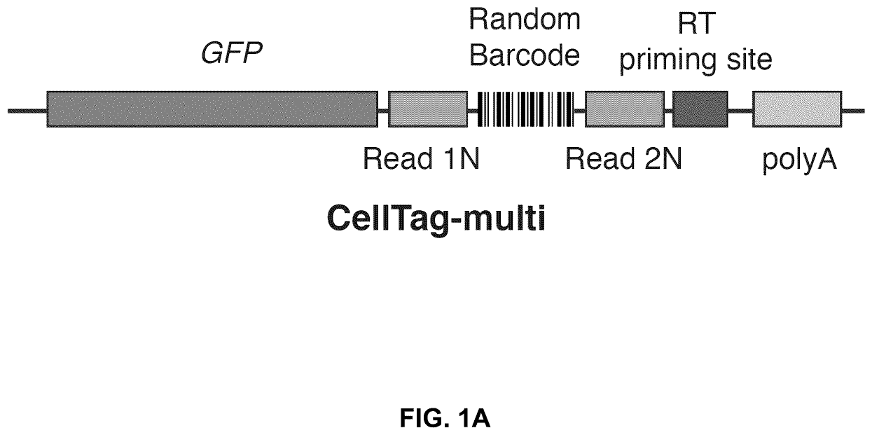

In some embodiments, the lineage tracing construct includes a reporter/GFP gene with specific modifications in the 3′ UTR to enable lineage tracing, as shown illustrated in FIG. 1A. The specific modifications in this aspect include a green fluorescent protein reporting sequence (GFP), a static random sequence used for lineage tracing (random barcode), 2 Nextera adapters flanking the random barcode sequence (Read 1N and Read 2N), and a reverse transcription (RT) priming site. In some embodiments, this sequence is packaged in lentiviral particles and inserted into cellular genomes via viral transduction. In various aspects, the lineage barcodes of the genetic construct provide unique labeling of each cell to facilitate lineage tracking. In other aspects, the Nextera adapters and RT priming site assist in the downstream capture of CellTags in genome-wide assays.

In other embodiments, the lineage tracing construct is a modification of the lineage tracing construct to provide for compatibility with other assays including, but not limited to, single-cell histone profiling (e.g. single-cell CUT&Tag) that typically require incubating intact nuclei with primary antibodies overnight, shown illustrated in FIG. 1B. The lineage tracing construct includes the green fluorescent protein reporting sequence (GFP), the static random sequence used for lineage tracing (random barcode), 2 Nextera adapters flanking the random barcode sequence (Read 1N and Read 2N), and reverse transcription (RT) priming site of the lineage tracing construct illustrated in FIG. 1A. In addition, the modified construct of FIG. 1B further includes a promoter sequence positioned between the end of the GFP coding sequence (CDS) and the start of the GFP Untranslated region (UTR) that houses the CellTag barcode and other adapters. Using in situ transcription through this promoter, we can boost the number of CellTag-containing RNA molecules in nuclei undergoing single-cell library preparation. This would be helpful for CellTag barcode capture in additional assays including, but not limited to, single-cell histone profiling (e.g. single-cell CUT&Tag), which often require incubating intact nuclei with primary antibodies overnight, likely leading to a significant loss of nuclear RNA (and hence CellTag RNA) due to diffusion. Non-limiting examples of suitable promoter sequences include T7/T5 sequences.

In various aspects, a method to prepare a modified genetic library is disclosed that makes use of at least one of the lineage tracing constructs disclosed herein. In some aspects, CellTag capture in 3′ scRNA-seq assays is performed wherein the CellTag-multi construct is inserted in the 3′ UTR of a transcribed gene. In some aspects, a protocol for CellTag capture in scATAC-seq assays is disclosed. In other additional aspects, a protocol for CellTag capture in additional assays including, but not limited to, single-cell histone profiling (e.g. single-cell CUT&Tag) is disclosed. In various other aspects, CellTag capture is performed on any cell assay that relies on poly-adenylated RNA, tagged fragments of cDNA, tagged fragments of DNA, or any combination thereof.

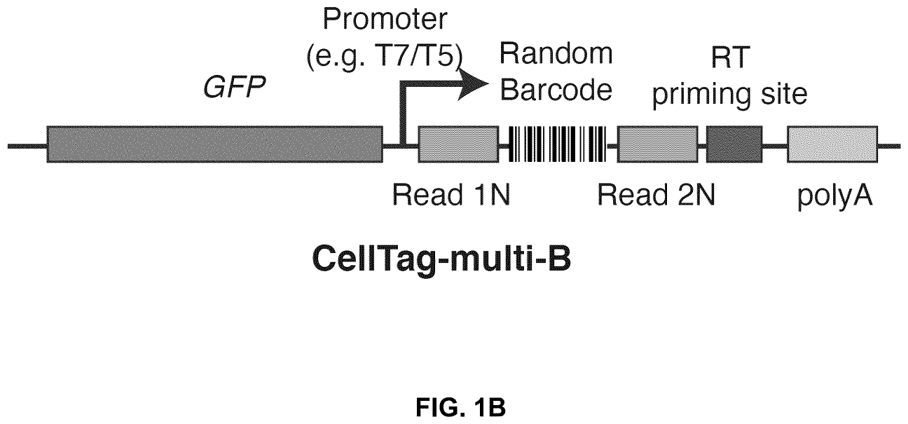

In one embodiment, shown illustrated in FIG. 2A, nuclei from cells labeled with the CellTag-multi library are isolated and Tn5 tagmentation is performed with ATAC protocol. A modified in situ RT step is then performed that uses the RT priming site to selectively reverse transcribe the entire CellTag-multi construct, including the lineage tracing barcode and the Nextera adapters into cDNA. Following this, these nuclei are loaded onto the 10× Genomics scATAC-seq chip according to the manufacturer's protocol, with one addition. In some embodiments, an in-GEM PCR primer for CellTag amplification is added to the cell suspension prior to loading on the 10×chip. During the GEM incubation step, single-cell barcoding oligos that are designed to label and amplify fragments of accessible chromatin also label and amplify CellTag cDNA, due to the presence of the Nextera adapter sequences in the construct. In some embodiments, the in-GEM PCR primer enables exponential amplification of the CellTags, while the rest of the library undergoes linear amplification. In some embodiments, the remainder of the prep is performed in accordance with the manufacturer's protocol. In this and other embodiments, the final sequencing library produced as described above contains single-cell barcoded fragments from both accessible genomes and CellTags and enables parallel assay of chromatin landscape and clonal identity.

In various aspects, the CellTag computational pipeline consists of a series of steps. First, CellTag-multi reads for each cell from all the sequencing data are collected using a known pattern of nucleotides specific to the CellTag construct. Next, a series of filtering, error correction, and allow-listing steps are performed to denoise the data. Finally, a cell-cell similarity graph is constructed based on each cell's CellTag signature and fully connected sub-components are identified, each of which is considered a clone.

In some embodiments, the method uses Tn5 transposase and Nextera adapter sequences to fragment the genome. In other embodiments, the method uses alternative transposases to fragment the genome. In various embodiments, any suitable method that fragments the genome in a functionally biased manner while also simultaneously tagging those fragments with known sequences may be used. Transposases, including but not limited to Tn5, can be loaded with custom sequences. In one aspect, as long as the sequences of the adapters are known, the technology can be modified to be compatible with any adapter with a known sequence.

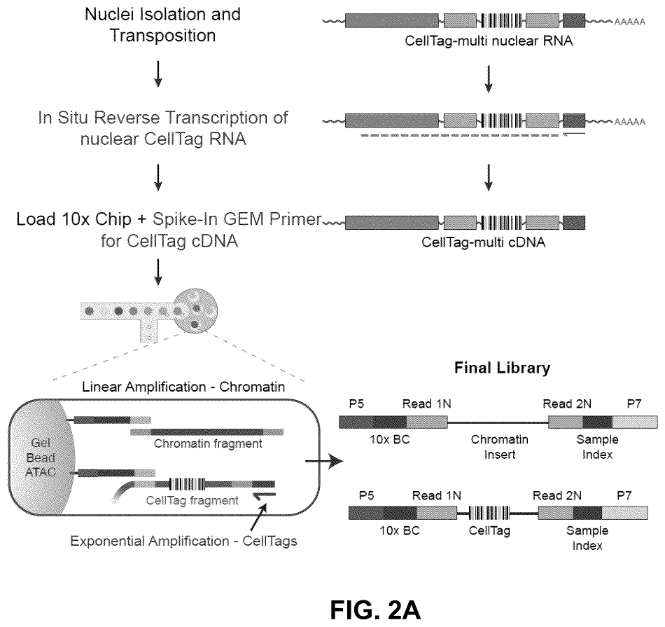

Measuring cell identity is central to understanding development, disease, and reprogramming. In some aspects, cell identity can be defined with three main pillars (FIG. 3). One pillar is phenotype and function (present), which includes morphology, location, neighbors, transcriptome, proteome, and function. The second pillar is lineage (past), which can include building a cellular taxonomy from developmental origins. The third pillar is cell state (future), which includes distinguishing between cell type and cell state.

In some aspects, computational approaches to measure cell identity are disclosed. In one aspect, the computational approach comprises Capybara, which measures cell identity and fate transitions. A detailed description of Capybara is provided in Kong, et al. 2022 (Cell Stem Cell. 2022 Apr. 7; 29(4): 635-649.e11. doi:10.1016/j.stem.2022.03.001) the content of which is incorporated by reference herein in its entirety. In some aspects, cell identity can be measured on a continuum. In some aspects, each single-cell identity represents a linear combination of all potential cell identities, using existing atlases as a reference. In some aspects, the methods include quadratic programming. In some aspects, Capybara accurately classifies discrete cell identity. In one aspect, Capybara captures hybrid cell identity. In one aspect, scRNA-seq is performed, which is used to validate hybrid cells using lineage tracing. In some embodiments, the majority of hybrid cells are monocyte-neutrophils. In another aspect, Capybara captures bistable hybrid states. In yet another aspect, Capybara captures bistable intermediates in addition to transition states. In some aspects, the methods dissect gene regulation of hybrid cell states, including, but not limited to, GNR inference and multi-omic lineage tracing.

In some aspects, CellTagging is performed, including cell barcoding to track clonally-related cells. In some aspects, simple lentiviral transduction can be performed to introduce the disclosed lineage tracing construct into cells to be studied. In some aspects, cells usually express about 3-4 CellTags per cell. In another aspect, CellTags are heritable. In another aspect, parallel capture of lineage information and cell identity can occur using the disclosed methods. In some aspects, over 70% of cells pass the indexing threshold.

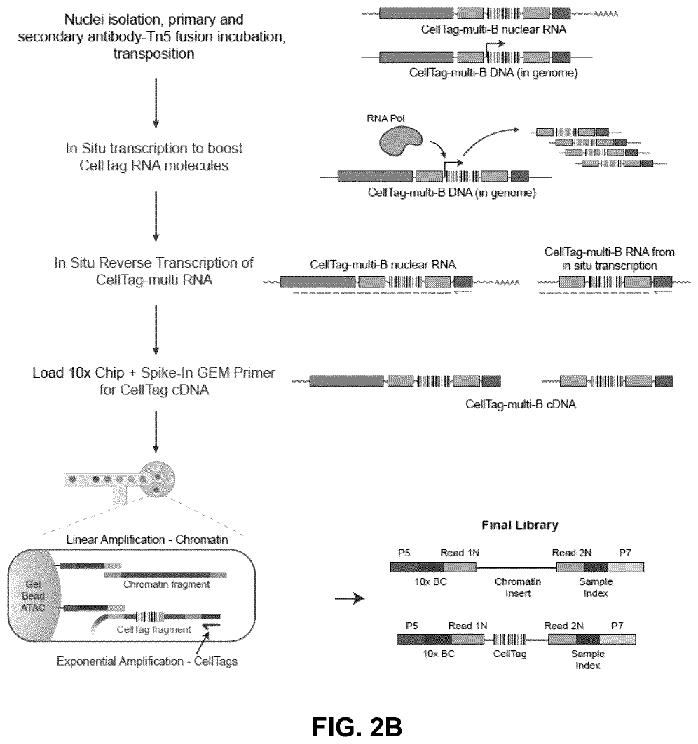

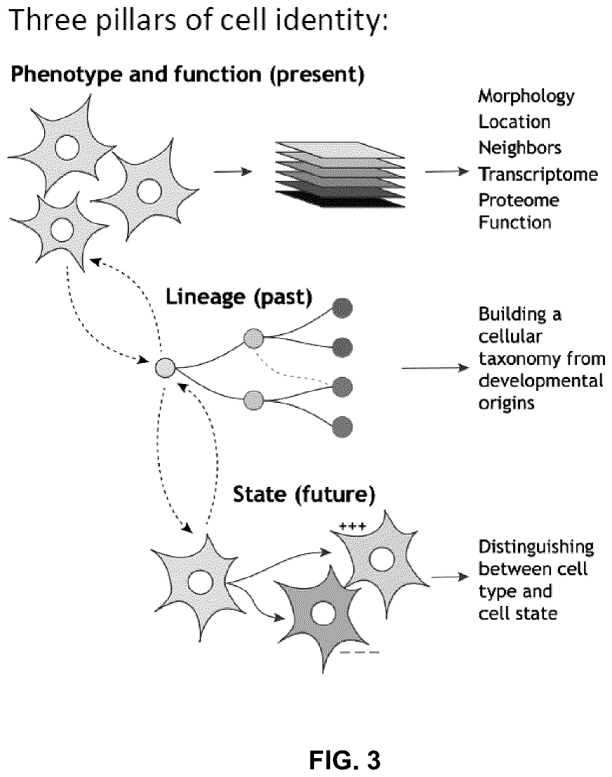

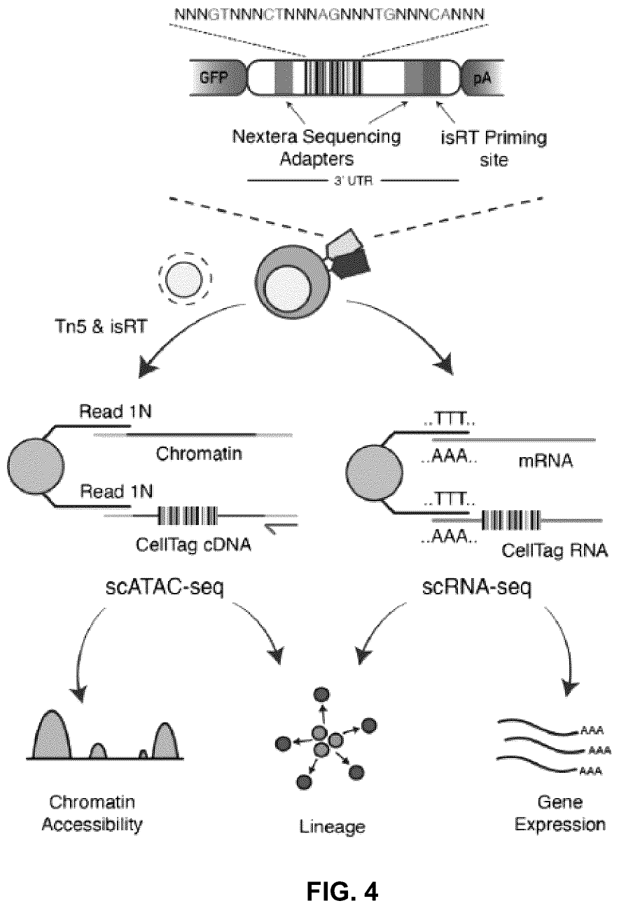

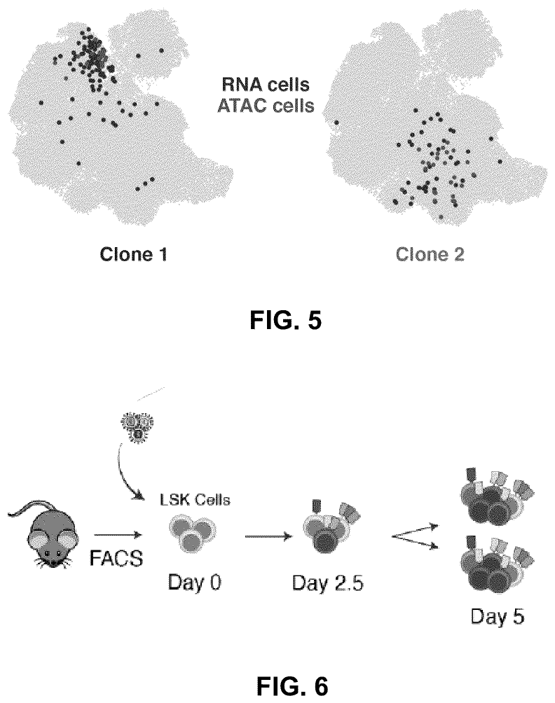

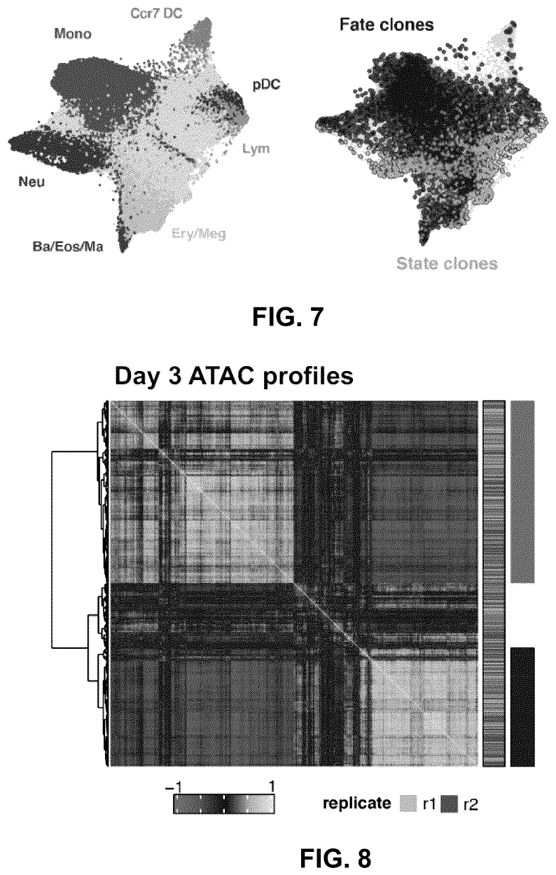

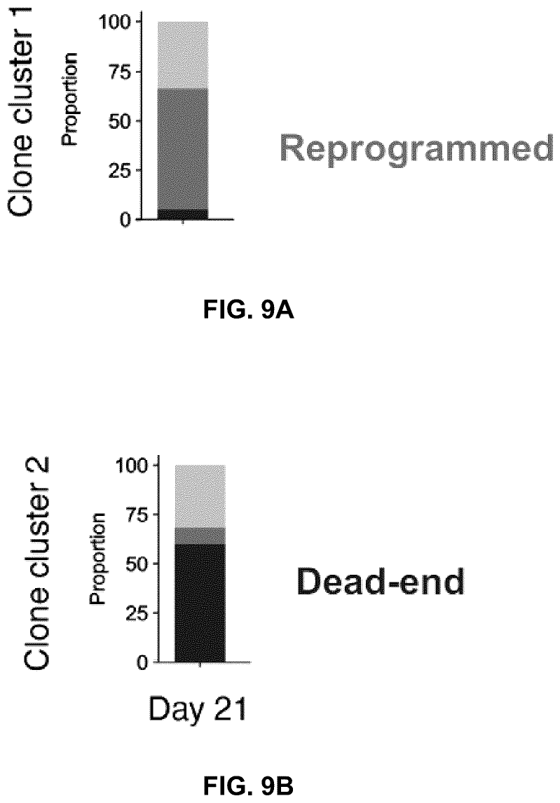

In some aspects, CellTag-ATAC-RNA methods are performed (FIG. 4), which can provide effective capture of chromatin accessibility and lineage information (FIG. 5). In another aspect, CellTag-ATAC-RNA methods that reconstruct state-fate relationships in hematopoiesis are performed (FIGS. 6 and 7). In another aspect, CellTag-ATAC-RNA methods interrogate iEP reprogramming (FIGS. 8 and 9). In some aspects, pooled libraries such as Addgene, various protocols, code, and tutorials with tools such as GitHub, data exploration and simulator from celltag.org, MightyMorphin CellTags, and CellTag-ATAC are incorporated in the disclosed methods.

In various aspects, the disclosed computational pipeline to measure cell identity, Capybara, is configured to capture hybrid states, representing fate transitions and bistable intermediates, as well as cell identities.

By way of non-limiting example, Capybara was used to identify impaired dorsal-ventral patterning during motor neuron programming. The addition of retinoic acid to motor neuron programming increased target cell yield. iEPs, a poorly defined cell type, were revealed to possess BEC-like potential.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in a genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desire to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of a pathway by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome

For example, the methods as described herein can comprise a method for altering a target polynucleotide sequence in a cell comprising contacting the polynucleotide sequence with a clustered regularly interspaced short palindromic repeats-associated (Cas) protein.

### Description of Multiomics CellTagging:

In various aspects, CellTagging is a system for lineage tracing that is compatible with a wide range of single-cell assays. In some aspects, CellTag-multi may be used for scRNA-seq and scATAC-seq lineage tracing. In other aspects, CellTag-multi may be rendered compatible with other single-cell assays after modification of the CellTaq-AT construct in the capture protocol. In various aspects, the CellTag-multi lineage tracing system consists of 3 components including the lineage tracing construct itself, a modified library preparation protocol that provides for CellTag capture in a wide variety of single-cell genomics assays, and a computational pipeline that provides for the identification of clones across single-cell data from multiple modalities.

CellTag-Multi Lineage Tracing Construct:

The lineage tracing construct consists of a reporter/GFP gene (GFP) with specific modifications in the 3′ UTR to enable lineage tracing (FIGS. 1A and 1B). As illustrated in FIG. 1A, in some aspects, these modifications include a static random sequence used for lineage tracing (random barcode), 2 Nextera adapters flanking this sequence (Read 1N and Read 2N), and a reverse transcription (RT) priming site. In other aspects, shown illustrated in FIG. 1B, the modifications to the reporter/GFP gene (GFP) in the 3′ UTR further include promoter sequence positioned between the 5′ end of GFP coding sequence (CDS) and the start of the GFP Untranslated region (UTR) that houses the CellTag barcode and other adapters. In some aspects, the lineage tracing construct sequence is suitable for packaging in lentiviral particles and insertion into cellular genomes via viral transduction. The lineage barcodes allow the unique labeling of each cell. The Nextera adapters and RT priming site assist in the downstream capture of CellTags in genome-wide assays.

Modified Library Preparation:

CellTag capture in 3′ scRNA-seq assays is accomplished by inserting the CellTag-multi or CellTag-multiB constructs disclosed herein in the 3′ UTR of a transcribed gene. For non-scRNA-seq single-cell assays, such as scATAC-seq, CellTag capture is challenging as these assays are designed to capture genomic fragments instead of transcripts. A protocol for CellTag capture in scATAC-seq is described below in one aspect but may be modified for use with other assays.

As illustrated in the flow chart of FIG. 2A, in some aspects nuclei from cells labeled with the CellTag-multi library (FIG. 1A), are isolated and Tn5 tagmentation is performed, according to the standard ATAC protocol. Then a modified in situ RT step is performed that uses the RT priming site to selectively reverse transcribe the entire CellTag-multi construct, including the lineage tracing barcode and the Nextera adapters into cDNA. The transcribed nucleus CellTag RNA is then loaded onto a 10× Genomics scATAC-seq chip according to the manufacturer's protocol, after the addition of an in-GEM PCR primer for CellTag amplification to the cell suspension prior to loading on the 10×chip. During the GEM incubation step, single-cell barcoding oligos that are designed to label and amplify fragments of accessible chromatin also label and amplify CellTag cDNA due to the presence of the Nextera adapter sequences in the construct. Additionally, the in-GEM PCR primer enables exponential amplification of the CellTags, while the rest of the library undergoes a linear amplification. The remainder of the sample prep is performed in accordance with the manufacturer's protocol. The final sequencing library produced as described above contains single-cell barcoded fragments from both accessible genomes and CellTags, enabling the parallel assay of chromatin landscape and clonal identity.

In other aspects, shown illustrated in the flow chart of FIG. 2B, nuclei from cells labeled with the CellTag-multiB library (FIG. 1B) are isolated, primary and secondary antibody-Tn5 fusion incubation, and transposition is performed. Then a modified in situ RT step is performed that uses the RT priming site to selectively reverse transcribe the entire CellTag-multi construct, including the lineage tracing barcode and the Nextera adapters into cDNA. The transcribed nucleus CellTag RNA is then loaded onto a 10× Genomics scATAC-seq chip according to the manufacturer's protocol, after the addition of an in-GEM PCR primer for CellTag amplification to the cell suspension prior to loading on the 10×chip. During the GEM incubation step, single-cell barcoding oligos that are designed to label and amplify fragments of accessible chromatin also label and amplify CellTag cDNA due to the presence of the Nextera adapter sequences in the construct. Additionally, the in-GEM PCR primer enables exponential amplification of the CellTags, while the rest of the library undergoes a linear amplification. The remainder of the sample prep is performed in accordance with the manufacturer's protocol. The final sequencing library produced as described above contains single-cell barcoded fragments from both accessible genomes and CellTags, enabling the parallel assay of chromatin landscape and clonal identity.

CellTag Computational Pipeline:

In various aspects, the computational pipeline consists of a series of steps. First, CellTag-multi reads for each cell from all the sequencing data are identified using a known pattern of nucleotides specific to the CellTag construct. Next, a series of filtering, error correction, and allow-listing steps are performed to denoise the data. Finally, a cell-cell similarity graph is built based on each cell's CellTag signature, and fully connected sub-components, each of which is considered a clone, are identified.

What is claimed is:

1. A genetic construct configured to label cells to capture cell lineage within at least one single-cell state assay, wherein the genetic construct comprises a reporter gene with modifications in the 3′ UTR, the modifications comprising:
a lineage tracing barcode comprising a static random sequence configured to uniquely label single cells and associated clones;
first and second flanking sequences at the 3′ and 5′ ends of the lineage tracing barcode, respectively; and
a reverse transcription priming site at the 5′ end of the second flanking sequence.

2. The genetic construct of claim 1, wherein the first and second flanking sequences each comprises a transposase.

3. The genetic construct of claim 2, wherein the first and second flanking sequences each comprises a Nextera adapter.

4. The genetic construct of claim 1, wherein the at least one single-cell state assay is selected from a single-cell transcriptomic assay, a genomic assay, an epigenomic assay, a multi-omics assay, and any combination thereof.

5. The genetic construct of claim 1, wherein the genetic construct is packaged into a lentiviral particle.

6. The genetic construct of claim 1, further comprising a promoter sequence positioned at the 3′ end of the first flanking sequence.

7. The genetic construct of claim 1, wherein the reporter gene is a green fluorescent protein (GFP) gene.

8. A method of labeling cells to trace cell lineage within at least one single-cell state assay, the method comprising inserting a genetic construct into the genome of a cell, the genetic construct configured to label cells to capture cell lineage within at least one single-cell state assay, wherein the genetic construct comprises a reporter gene with modifications in the 3′ UTR, the modifications comprising:
a lineage tracing barcode comprising a static random sequence configured to uniquely label single cells and associated clones;
first and second flanking sequences at the 3′ and 5′ ends of the lineage tracing barcode, respectively; and
a reverse transcription priming site at the 5′ end of the second flanking sequence.

9. The method of claim 8, wherein the genetic construct is inserted into the genome of the cells by viral transduction.

10. The method of claim 8, wherein the cell lineage is traced using scRNA-seq or scATAC-seq lineage tracing.

11. The method of claim 8, wherein the first and second flanking sequences each comprises a transposase.

12. The method of claim 8, wherein the first and second flanking sequences each comprises a Nextera adapter.

13. The method of claim 8, wherein the at least one single-cell state assay is selected from a single-cell transcriptomic assay, a genomic assay, an epigenomic assay, a multi-omics assay, and any combination thereof.

14. The method of claim 8, wherein the genetic construct is packaged into a lentiviral particle.

15. The method of claim 8, wherein the genetic construct further comprises a promoter sequence positioned at the 3′ end of the first flanking sequence.

16. The method of claim 8, wherein the reporter gene of the genetic construct is a green fluorescent protein (GFP) gene.
